(19) United States
(12) Patent Application Publication
(10) Pub. No.: US 2006/0286572 A1
(43) Pub. Date: Dec. 21, 2006

(54) METHOD FOR PRODUCING CHEMICALLY SYNTHESIZED AND IN VITRO ENZYMATICALLY SYNTHESIZED NUCLEIC ACID OLIGOMERS

(76) Inventor: David E. Kohne, San Diego, CA (US)

Correspondence Address: WESLEY B. AMES, 7031 LOS VIENTOS SERENOS, ESCONDIDO, CA, 92029, US

(21) Appl. No.: 11/383,198

(22) Filed: May 13, 2006

Related U.S. Application Data
(60) Provisional application No. 60/681,524, filed on May 16, 2005.
(60) Provisional application No. 60/681,426, filed on May 16, 2005.

(51) Int. Cl. C12Q 1/68; C07H 21/04; C12P 19/34

(52) U.S. Cl. 435/6; 435/91.2; 536/24.3

(57) ABSTRACT

A method is described for producing chemically synthesized and in vitro enzymatically synthesized oligomer and other nucleic acid molecules which are associated with less nucleotide sequence damage than prior art chemically synthesized and biologically synthesized oligomers and other nucleic acid molecules, along with the use of such improved oligomers and other nucleic acid molecules to improve oligomer application results and further application results.

### RELATED APPLICATIONS

This application claims the benefit of Kohne, U.S. Provisional Application 60/681,524, filed May 16, 2005, and Kohne, U.S. Provisional Application 60/681,426, filed May 16, 2005, each of which is hereby incorporated by reference in its entirety.

### FIELD OF THE INVENTION

The invention relates overall to: chemically synthesized nucleic acid oligomer preps of all kinds; in vitro enzymatically synthesized nucleic acid preps of all kinds which are produced using a chemically synthesized nucleic acid such as for example an oligomer primer or ligation molecule; the applications these chemically synthesized and in vitro enzymatically synthesized nucleic acid preparations are associated with; as well as applications which utilize oligomer application results for further purposes. The invention relates to the quality of such synthesized nucleic acid molecules of all kinds, and the degree of nucleic acid molecule homogeneity as well as the degree of nucleotide sequence damage which is associated with such synthesized oligomer molecule preps. More specifically, the invention is related to the quality of, and the degree of damage associated, with the 3′ or 5′ end nucleotide sequence region of chemically synthesized oligomer preparations, and to the quality of, and the degree of damage associated with, the 5′ end nucleotide sequence region of in vitro enzymatically synthesized nucleic acid preparations.

The invention also relates to the use of invention-improved oligomer preparations to produce improved oligomer application results, as well as the use of invention-improved oligomer application results in further applications to produce improved further application results. Such further applications are very broad and include, but are not limited to, Data Mining analysis, Systems Biology analysis, and various aspects of drug discovery, validation, and use.

### BACKGROUND OF THE INVENTION

The following discussion is provided solely to assist the understanding of the reader, and does not constitute an admission that any of the information discussed or references cited constitute prior art to the present invention.

The vast majority of chemically synthesized oligomer molecules are synthesized on a solid support, and the chemical synthesis is initiated at the 3′ end of the intended oligomer (1-5). The vast majority of in vitro enzymatically synthesized nucleic acid molecule preps are produced by enzymes which utilize chemically synthesized oligomer molecules as primers (2,6,7). Here the synthesis is initiated at the 5′ end of the intended enzymatically synthesized molecule and at the 3′ end of the chemically synthesized oligomer primer. The final enzymatically synthesized nucleic acid molecule then, possesses a chemically synthesized oligomer molecule at its 5′ end, and the rest is enzymatically synthesized.

It is well known that in vitro enzymatically synthesized nucleic acid molecules are associated with far less nucleotide sequence damage or errors than are chemically synthesized oligomer molecules (8). Such nucleotide sequence damage or errors include, but are not limited to, misincorporated nucleotides or bases, damaged or modified nucleotides or bases, deletions, and apurinic or abasic sites. Therefore the 5′ end primer portions of any in vitro enzymatically synthesized nucleic acid molecule preps which are produced using chemically synthesized oligomer molecules will be associated with significantly more oligomer damage than the non 5′ end portions of the same enzymatically synthesized nucleic acid molecules.

For convenience herein, a chemically synthesized nucleic acid oligomer will be termed a CS oligomer. Further, an in vitro enzymatically synthesized nucleic acid molecule, which consists of a 5′ end CS oligomer primer portion and an enzymatically synthesized portion, is termed an ES molecule. Here the portion of the ES molecule, which is enzymatically synthesized, is termed the ES portion. The nucleotide length of a CS oligomer can range from 2 to 150 nucleotides long or longer. The nucleotide length of the ES portion of an ES molecule can range from 2 to 10,000 or more nucleotides long.

It is also well known that for a CS oligomer prep the nucleotide sequence damage associated with the 3′ end portions of the CS oligomer molecules in the oligomer prep, is much greater than for the rest of the CS oligomer molecule nucleotide sequence (9,10). Further, it has been reported that most of this damage occurs during the first 5 to 8 synthesis steps, i.e. most of the damage is associated with the first 6 to 9 bases at the 3′ end of the CS oligomer molecule (9). Reports indicate that about 60% of the detected damage is associated with the first 6 3′ end nucleotide positions, and that about 73% of the detected damage is associated with the first 9 3′ end nucleotide positions. This greater 3′ end damage is reported to be to be related to the proximity of the 3′ end to the solid surface and to inefficiencies in the synthesis process reactions caused by steric effects.

Reports also indicate that during the synthesis of a 25 nucleotide long CS oligomer, the last 4 5′ end bases were not associated with any nucleotide sequence damage. For the standard synthesis of CS oligomers reports indicate the following. (a) the nucleotide sequence damage associated with the 3′ end portion of a CS oligomer is much greater than that associated with the 5′ end or middle portions of the CS oligomer. (b) the nucleotide sequence damage associated with the 5′ end portion is much less than that associated with the middle portion of the CS oligomer. This pattern is believed to be reversed for the 5′ to 3′ synthesis of oligomers.

The above quoted nucleotide sequence damage measurements were obtained by the cloning method described in reference 9, and can be used for evaluating the nucleotide sequence damage for oligomer preparations. The extent of nucleotide sequence damage associated with an oligomer preparation can also be determined using the methods described in the U.S. Provisional Patent number US60/681,426 entitled “Method for Producing Improved Oligomer Functional Homogeneity and Functional Characteristic Information and Results”, which is incorporated herein by reference in its entirety.

It is also known that nucleotide sequence damage in the 3′ end portion of a CS oligomer can significantly affect the effectiveness of the oligomer application the CS oligomer is used in. For example, nucleotide sequence damage associated with the 3′ end portion of a CS oligomer primer can significantly affect the efficiency of in vitro enzymatic synthesis of nucleic acids. As an example, the PCR amplification efficiency is significantly influenced by nucleotide sequence damage associated with the 3′ end portion of a CS oligomer PCR primer (11). Such 3′ end associated nucleotide sequence damage would have a deleterious effect on most if not all CS oligomer applications.

### SUMMARY OF THE INVENTION

The present invention addresses the difficult problems resulting from oligonucleotide sequence damage by providing methods for preparing enhanced quality oligomer preparations. In addition the invention concerns methods for obtaining improved information in applications using such improved oligomer preparations, and further applications incorporating the resulting improved information.

Generally, the methods for producing oligonucleotide preparations having improved sequence quality involve chemically synthesizing a desired sequence using immobilized starting chains, which can have moieties susceptible to cleavage at or near their ends. A desired nucleotide sequence synthesized on the ends of the starting chains will then be of higher sequence quality than if the desired sequence were synthesized with direct or close attachment to a solid phase surface.

Thus, in a first aspect the invention concerns a method for chemically synthesizing a nucleotide oligomer preparation significantly improved in nucleotide sequence quality by sequentially coupling in order chain moieties of a wanted portion or wanted polymer (WP) nucleotide sequence to an immobilized unwanted portion or unwanted polymer (UP). The unwanted polymer includes, in order, an attachment chain which is Y chain moieties in length, and a spacer chain which is Z chain moieties in length. Optionally the unwanted chain also includes W cleavage site compound (CSC) chain moieties. A chain which includes unwanted polymer and wanted polymer (a UP+WP chain) is thus formed. In the unwanted chain, Y, Z, and W (when present) are each independently equal to or greater than 1.

In many applications, it is desirable to have WP removed from the UP; this in some embodiments, the method also includes cleaving (preferably specifically cleaving) WP from UP at one or more cleavage site chain moieties, and/or the method also includes separating WP from UP.

In particular embodiments the method includes preparation of the unwanted polymer, and thus includes sequentially coupling Z nucleotides, nucleotide analogs, nucleotide substitutes, or a combination thereof to a surface immobilized attachment chain Y chain moieties in length of nucleotides, nucleotide analogs, nucleotide substitutes, or a combination of such molecules to form an attachment spacer chain, and coupling to the attachment spacer chain W cleavage site compounds (CSC) producing the unwanted polymer (UP).

In embodiments involving cleavage of the WP from the UP, the cleavage is performed by subjecting the UP+WP chain to a condition or combination of two or more conditions which promote the specific cleavage of the UP+WP chain at the cleavage site compound location to convert the intact UP+WP finished oligomer chain to separate unattached UP and WP oligomer molecules. Such cleavage is usually performed using selected chemical conditions, but can also be performed in many cases using enzymatic cleavage.

In certain embodiments, the UP+WP chain or the WP is subjected to one or more conditions which promotes the removal of all or essentially all chemical synthesis process related protective and/or modifier chemical groups from the WP and/or WP +UP and/or WP and UP; the WP is purified away from the UP and/or other unwanted non-WP molecules and solid components; the WP (e.g., WP separated from UP) is processed to produce a purified preparation of WP enriched for WP oligomer molecules of the intended nucleotide length N; Y is 1 or 2; Z is equal to 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 nucleotides, nucleotide analogs, or nucleotide substitutes, or combinations thereof; Z is at least 24 nucleotides, nucleotide analogs, or nucleotide substitutes, or combinations thereof; Z is 2-50, 4-40, 6-30, 10-30, 15-30 inclusive, or is any integer value in such range; W is 1, 2, 3, 4, 5, 6, or more, or 1-10, 1-7, 1-5; the WP is 6-50 nucleotides, nucleotide analogs, and/or nucleotide substitutes in length; the 3′ end nucleotide of a 3′ to 5′ synthesized and cleaved and deprotected WP oligomer, or the 5′ end nucleotide of a 5′ to 3′ synthesized and cleaved and deprotected WP, possesses the proper chemical composition and configuration so that the WP oligomer can be directly used in the intended oligomer application without further modification; the starting nucleotide, nucleotide analog, or nucleotide substitute molecules are attached to the surface by a chemical bond which is not cleaved under any of the conditions used for the production of the purified crude WP oligomer prep; the starting nucleotide, nucleotide analog, or nucleotide substitute molecules are attached to the surface by a chemical bond which is readily cleavable under one or more of the conditions used for the production of the purified crude WP oligomer prep;

Likewise in certain embodiments, the starting chain of nucleotide or nucleotide substitution molecules is attached to the surface by a chemical bond which does not cleave under any of the conditions used for the UP or WP synthesis or processing or use, and a cleavage site compound (CSC) molecule is not incorporated into the UP oligomer chain, such that after deprotecting the UP+WP oligomer molecules and removing all or essentially all of the chemical synthesis process related protective and modifier chemical groups from the UP+WP oligomer molecule, the UP+WP oligomer molecules remain stably attached to the surface of the synthesis support. In further embodiments of such stably attached oligomers, the oligomer preparation is used in an oligomer application with the UP+WP chains in the immobilized state.

In particular embodiments. a modified or unmodified RNA or DNA WP oligomer is produced (which may be immobilized or free); a chimeric WP oligomer consisting of a combination of modified or unmodified RNA and modified or unmodified DNA is produced (which may be immobilized or free); the starting chain contains at least one, 2, 3, 4, or 5 nucleotide or nucleotide analog or nucleotide substitute molecules, or a combination of 1 or 2 or more of such nucleotide, nucleotide analog, nucleotide substitute molecules; the UP contains at least 2, 3, 4, 5, 7, 10, 15, 20, 30, 2-30, 4-40, 6-30, 10-30, 10-20, or 20-30 chain moieties.

In a related aspect, the invention concerns a method for producing improved oligomer primer dependent in vitro enzymatically synthesized RNA or DNA molecules, by utilizing one or more oligonucleotides from an improved oligomer preparation prepared according to an embodiment of the preceding aspect as primers in an in vitro enzymatic synthesis system to produce improved enzymatically synthesized RNA (ES RNA) or enzymatically synthesized DNA (ES DNA) molecules, e.g., using PCR, RT-PCR, or other such processes.

In another aspect, the invention concerns an improved oligonucleotide preparation which contains a set of chemically synthesized immobilized oligonucleotide chains which include an unwanted polymer chain (UP) at least 3 chain moieties in length, and a wanted polymer chain (WP) consisting essentially of a desired sequence of nucleotides, where the unwanted chain (UP) includes at least one cleavage site moiety proximal to said wanted chain.

In particular embodiments, the oligonucleotide preparation is sufficiently improved such that the first 10 nucleotides or nucleotide residues of the wanted polymer chains (or of the N-X fraction of WP chains) contain an average density of damaged nucleotide sequence sites which is no more than 0.9, 0.8, 0.7, 0.6, 0.5, 0.4, 0.3, 0.2, or 0.1 times the average density of damaged nucleotide sequence sites in the first ten nucleotide or nucleotide analogs in the same sequence produced in the absence of unwanted polymer chains. In certain embodiments, the density of damaged nucleotide sequence sites is over the first 5, 7, 10, 15, 20, 25 such sites or over the entire WP. Alternatively, in particular embodiments, the WP or N-X fraction of the WP (or a portion as just specified) contains on average, no more than 1.0, 0.9, 0.8, 0.7, 0.6, 0.5, 0.4, 0.3, 0.2 or 0.1 percent nucleotide sequence damage.

In certain embodiments, the unwanted polymer chain is 2, 3, 4, 5, 7, 10, 15, 20, 30, 2-30, 4-40, 6-30, 10-30, 10-25, 10-20, or 20-30 chain moieties in length; the wanted polymer chain is 10-100, 10-70, 10-50, 15-50, 20-40, 20-30 chain moieties in length; the oligomer preparation, oligomers, or WP are as described for an embodiment of an above aspect.

Another aspect concerns a kit for preparing an oligonucleotide preparation, where the kit includes at least one solid phase medium to which are attached a plurality of immobilized oligomers each having an attached terminus and a free terminus. The immobilized oligomers include in order: an attachment moiety, a spacer chain, and a cleavage site moiety. The free terminus of each of the chains is functionalized or is chemically suitable for functionalizing with a functional group for extending the oligomer with a plurality of chain moieties.

In particular embodiments, the solid phase medium is or includes beads, nanoparticles, microparticles, a chip, a plate, a filter; the kit also includes instructions for using the kit and/or the solid phase medium; the kit also includes a buffer solution chemically compatible with the oligomers; the kit also includes separate quantities of at least 2, 3, or 4 different compounds suitable for incorporation in an oligomer, such as phosphoramidites corresponding to 2, 3, or 4 naturally occurring ribonucleotides or deoxyribonucleotides; the kit is a single use kit.

Also in certain embodiments, the immobilized oligomers include, consist essentially of, or consist of 2, 3, 4, 5, 7, 10, 15, 20, 30, 2-30, 4-40, 6-30, 10-30, 10-25, 10-20, 20-30, 4-50, or 6-40 chain moieties. In particular embodiments, the immobilized oligomer is as described for an aspect above.

The production of improved oligomer preparations also provides improved results and information resulting from use of such preparations in various oligomer applications. Thus, in another aspect the invention provides a method for producing improved oligomer application results by utilizing a synthetic oligomer preparation produced by a method as described in the first aspect above or an embodiment thereof in an oligomer application, thereby providing improved zero order oligomer application results.

Desirably, the wanted polymer (WP) utilized in the oligomer application is of significantly enhanced quality. Thus, in particular embodiments, the first 10 nucleotides or nucleotide residues of the wanted chains contain an average density of damaged nucleotide sequence sites which is no more than 0.9, 0.8, 0.7, 0.6, 0.5, 0.4, 0.3, 0.2, or 0.1 times the average density of damaged nucleotide sequence sites in the first ten nucleotide or nucleotide analogs in the same sequence produced in the absence of unwanted chains. Also in particular embodiments, the just-specified limits on average density of damaged nucleotide sequence sites is for the first 5, 10, 15, 20, or 25 nucleotides or nucleotide analogs in the wanted chains, for the entire wanted chains. Similarly, in particular embodiments, the specified average density of damaged nucleotide sequence sites is for the N-X fraction molecules of the WP portion, as compared to the N-X fraction molecules of an oligomer preparation prepared in the absence of UP. Alternatively, the first 5, 10, 15, 20, 25, or all nucleotide sequence of the WP (or the N-X fraction molecule of the WP) contain on average no more than 0.8, 0.7, 0.6, 0.5, 0.4, 0.3, 0.2, or 0.1 percent damaged sites. The specified limits on damaged nucleotide sites in WP also apply to embodiments of the other aspects herein involving production of oligomer preparations according to the present invention.

The information and results from the zero order oligomer application are commonly applied in further oligomer applications. Therefore, in further embodiments, the results from the zero order oligomer application or results derived from the zero order application are used in a further oligomer application, e.g., a first order, second order, third order, or higher than third order oligomer application.

In exemplary embodiments, the zero order application is or includes an application such as a) a primer application, b) a hybridization probe application, c) a gene expression analysis application, d) a site directed mutagenesis application, e) a microarray hybridization capture probe application, f) a cloning application, g) a gene synthesis application, h) a fluorescent-labeled oligomer application, i) a mutation or single nucleotide polymorphism detection application, j) a nucleic acid sequencing application, k) a nucleic acid standard application, or 1) a production of an oligomer primer dependent in vitro enzymatically synthesized RNA (ES RNA) or DNA (ES DNA) molecule application.

Similarly, in a related aspect, the invention concerns a method for improving an oligomer application which involves incorporating data produced directly or indirectly using an improved oligomer preparation produced by a method according to an aspect above in that oligomer application.

In particular embodiments, the data is produced by or used in an oligomer application as specified in the preceding aspect (commonly but not necessarily zero order applications).

In further embodiments, the data is produced by or used in an oligomer application such as a) a gene expression or gene expression comparison analysis application, b) a nucleic acid ligation based assay or procedure application, c) an application involving identification of one or more expressed particular genes or one or more gene expression profiles (e.g., genes or gene expression profiles which have properties which are characteristic or unique or nearly unique for one or more particular disease, pathologic state, or normal states) such as a Serial Analysis of Gene Expression (SAGE) or other clone counting analysis, d) a DNA or RNA probe diagnostic assay, e) a data mining analysis, f) an siRNA or miRNA or other regulatory RNA assay, or g) an siRNA or miRNA therapeutic application. Commonly but not necessarily the just-listed applications are first order oligomer applications.

Likewise, in particular embodiments, the data is produced by or used in an oligomer application such as a) a systems biology analysis application, b) a drug or bioactive compound or other compound or biomarker discovery and identification application (e.g., a drug discover compound screening, hit validation, or lead compound identification application); or c) a regulatory pathway discovery analysis application. Commonly but not necessarily the just-listed applications are second order oligomer applications.

Again similarly, in particular embodiments, the data is produced by or used in an oligomer application such as applications that include a) at least one method for producing improved drug or bioactive molecule or biomarker clinical study candidate selection results, b) at least one method for producing improved drug or bioactive molecule or biomarker clinical study evaluation results, c) at least one method for producing improved drug or bioactive molecule or biomarker manufacturing and QC/QA results, d) at least one method for producing improved drug or bioactive molecule or biomarker or other product market segment selection process results, e) at least one method for producing improved drug or bioactive molecule or biomarker or other product prescription and use in the patient results, f) at least one method for producing improved drug or bioactive molecule or biomarker efficacy or safety in the patient, g) at least one method for producing improved drug or bioactive molecule or biomarker or other product toxicological characteristic results, h) at least one method for producing improved disease or pathology state prognosis prediction results, or i) at least one method for producing improved disease or pathology state prognosis prediction after drug or bioactive molecule or other product treatment.

In a related aspect, the invention concerns a method for producing improved oligomer application information and results for an oligomer application, herein termed a zero order application, which directly utilizes the present invention-improved oligomer preparations, where the method includes using a method as described in an aspect above for producting improved oligomer preparations, and utilizing the improved oligomer preparation in a zero order application thereby producing one or more improved zero order application information and results.

In certain embodiments, the zero order application includes at least one of the following: a) a primer application; b) a hybridization probe application; c) a gene expression analysis application; d) a site directed mutagenesis application; e) a microarray hybridization capture probe application; f) a cloning application; g) a gene synthesis application; h) a fluorescent-labeled oligomer application; i) a mutation or single nucleotide polymorphism detection application; j) a nucleic acid sequencing application; k) a nucleic acid standard application; and l) a production of an oligomer primer dependent in vitro enzymatically synthesized RNA (ES RNA) or DNA (ES DNA) molecule application.

Similarly, in another related aspect, the invention provides a method for producing improved information and results for a first order oligomer application, where the method includes a) producing improved zero order application results according to the preceding aspect, and b) utilizing all or part of the improved zero order application results in a first order application, thus producing one or more improved first order application information and results.

In particular embodiments, the first order application is or includes one of the following applications: a) a gene expression or gene expression comparison analysis application; b) a nucleic acid ligation based assay or procedure application; c) identification of one or more expressed particular genes or one or more gene expression profiles which have properties which are characteristic or unique or nearly unique for one or more particular disease, pathologic or normal states, d) a DNA or RNA probe diagnostic assay; e) a data mining analysis of any kind; and f) an siRNA or miRNA or other regulatory RNA assay.

A further related aspect of the invention concerns a method for producing improved information and results for a second order oligomer application, where the method involves producing improved first order application information and results as described in the preceding aspect, and utilizing all or part of the improved first order application results in a second order application, producing one or more improved second order application information and results.

Similarly to the preceding aspect, in particular embodiments the second order application includes or is one or more of the following applications: a) a systems biology analysis application; b) a drug or bioactive compound or other compound or biomarker discovery and identification application; and c) a regulatory pathway discovery analysis application.

In a further related aspect, the invention concerns a method for producing improved information and results for a higher order application, where the method includes producing improved lower order application information and results, e.g., according to any of the three preceding aspects, and utilizing all or part of the improved lower order application information and results in a higher order application, producing one or more improved higher order application information and results.

In particular embodiments, the lower order application includes or is one or more of a zero order application, a first order application, a second order application, a third order application, and a higher than third order application.

In certain embodiments, the higher order application includes or is one or more of the following: a) one or more methods for producing improved drug or bioactive molecule or biomarker clinical study candidate selection results; b) one or more methods for producing improved drug or bioactive molecule or biomarker clinical study evaluation results; c) one or more methods for producing improved drug or bioactive molecule or biomarker manufacturing and QC/QA results; d) one or more methods for producing improved drug or bioactive molecule or biomarker or other product market segment selection process results; e) one or more methods for producing improved drug or bioactive molecule or biomarker or other product prescription and use in the patient results; f) one or more methods for producing improved drug or bioactive molecule or biomarker efficacy or safety in the patient; g) one or more methods for producing improved drug or bioactive molecule or biomarker or other product toxicological characteristic results; h) one or more methods for producing improved disease or pathology state prognosis prediction results; and i) one or more methods for producing improved disease or pathology state prognosis prediction after drug or bioactive molecule or other product treatment.

In still another aspect,the invention concerns an improved artificial gene which includes one or more improved CS oligomer sequences (prepared as described herein) or sequences derived therefrom. Such artificial gene may have an intended sequence of all or part of a natural gene or cDNA form of such natural gene sequence, or may have an intended sequence which is an artificial sequence. Such artificial genes will usually be longer than oligomer length, e.g., greater than 400 chain moieties, at least 500, 600, 800, 1000, 1500, or 2000 chain moieties. Generally such artificial gene codes for a biologically active compound, e.g., an RNA or polypeptide. Also, often such artificial genes will include sequences for regulation of transcription and/or translation.

In the context of an oligomer, the terms “chain moiety” and “backbone moiety” interchangeably refer to a portion of the oligomer which results from incorporation of a molecular species in the chain. Unless clearly indicated to the contrary, such chain moieties may include nucleotides, nucleotide analogs, and nucleotide substitutes.

As used in connection with oligomer preparations, the terms “chemically synthesizing” refers to the process of covalently linking moieties including nucleotides and/or nucleotide analogs in the oligomer chain, but not using enzymes in that process.

In reference to separation of an oligomer chain into two or more chain portions, the term “cleaving” refers to the breaking of one or more bonds which form part of the backbone of the chain creating two or more separate chain portions. Thus, “specifically cleaving” refers to cleavage which occurs predominantly (and preferably exclusively or nearly exclusively) at a particular defined site or sites in the oligomer chain. Such cleavage may, for example, be performed enzymatically (e.g., using a nuclease) or non-enzymatically (e.g., using non-enzymatic chemical conditions).

Herein the extent of nucleotide sequence damage associated with the first synthesized end of a CS oligomer molecule is termed the “Damaged nucleotide Site Density”, or DSD for an oligomer prep. The DSD reflects the average number of damaged nucleotide sites per nucleotide for an oligomer molecule prep. For reference 9: the DSD value for the first five nucleotides added to a growing oligomer nucleotide chain is about four times greater than the DSD for the second five nucleotides added to the same growing chain; and the DSD value for the first ten nucleotides added to a growing oligomer chain is about three to four times greater than the DSD for the second ten nucleotides added to the same growing oligomer chain. Such sequence damage includes insertions, deletions, and damage to the structure of a nucleotide.

In reference to nucleotide sites in an oligomer, reference to the “first nucleotide” or “first nucleotide position” or site, or first 10 nucleotides positions or sites is defined with reference to the synthesis direction from the particular reference point (e.g., the first nucleotide position in a WP), such that the first nucleotide incorporated in the growing chain is the first nucleotide, and so on for the specified number of nucleotides. Thus, the counting may proceed in the 5′ to 3′ direction or 3′-5′ direction depending on the direction of synthesis.

As used herein the term “immobilized oligonucleotide” and like term such as immobilized polymer, immobilized chain, and immobilized oligomer refer to such chains which are attached to a solid phase medium in such manner that the attachment is stable under the relevant conditions. In many cases, the attachment is a covalent bond linkage.

The term “intended nucleotide length”, intended oligomer length”, “intended chain length” and like terms refer to the design length for a particular chain. It is the length which all chains would have in a perfect synthesis.

Herein, the terms “nucleoside” and “nucleotide” refers to one or more of the naturally occurring nucleosides or nucleotides, as for example the naturally occurring ribo- and deoxyribo- nucleosides and nucleotides of all kinds.

The terms “modified nucleoside” and “modified nucleotide”, and “nucleoside analogs” and “nucleotide analogs” are used interchangeably, and refer to chemically modified, non-naturally occurring nucleosides and nucleotides of any kind. The chemical modification may, for example, be at the base, sugar, and/or linkage portions of a nucleotide or nucleoside. Examples of such analogs include without limitation methylphosphonate nucleotide analogs, phosophorothioate nucleotide analogs, phosphorodithioate nucleotide analogs, peptide nucleic acid, locked nucleic acid, 2′-halo-modified nucleotides, 2′-alkyl-modified nucleotides, as well as other modified nucleosides and nucleotides

The terms “nucleoside substitute” and “nucleotide substitute” refer to one or more chemical compounds of any kind which are not naturally occurring or modified nucleoside or nucleotide compounds but which are incorporated in the backbone of a oligomer. Examples include sugars, and peptides, amino acids, and lipids, as well as other chemical compounds and combinations of these compounds. One of skill in the art will be aware that a large number of different modified and substitute nucleoside and nucleotide compounds exist which may be suitable for use in the invention.

Unless clearly indicated to the contrary, the terms “nucleotide oligomer”, “oligonucleotide”, and “oligomer” are used interchangeably to refer to covalently linked chain molecules at least 4 chain moieties in length, which principally contain nucleotides and/or nucleotide analogs in the backbone of the chain, but which may also contain one or more nucleotide substitutes (generall a relatively small number) such as sugars and the like in the chain backbone. Such oligomers may be up to 400 chain moieties (e.g., nucleotides or nucleotide analogs) in length, e.g., 6-25, 10-30, 15-50, 10-50, 25-50 50-100, 100-150, 150-200, 200-300, 300-400 chain moieties.

As used herein, the terms “oligomer preparation” and “nucleotide oligomer preparation” are used interchangeably to refer to set or population of chemically synthesized chains principally containing nucleotides and/or nucleotide analogs, but which may also contain one or more nucleotide substitutes. In most cases, the set will include a large number of such chains, e.g., at least 100, 1000, 10,000, 1,000,000, 10⁷, 10⁸, 10¹⁰, or more, even much more.

In the present context, the term “oligomer application” and like terms refer to a process which includes the direct or indirect use of an oligomer(s), information about such oligomer(s), or information or results produced directly or indirectly from a method using such oligomer(s) or information or results to produce particular information and/or results and/or compositions. Thus, an “improved oligomer application” is one which is better than a reference method in at least one characteristic, e.g., produces higher quality information and/or results and/or compositions, and/or is faster and/or easier to perform without sacrifing the quality of the information and/or results and/or compositions.

In the context of oligomer sythesis, the term “protective group” has its convention meaning, referring to groups attached to a moiety during or in preparation for the synthesis process to prevent unwanted reactions from occurring, e.g., during subsequent coupling steps or to prevent addition of additional sub-units. Normally such protective groups are removed or reacted to remove the group or modify it to a desired product (e.g., using particular chemical or light exposure conditions).

The term “purify” in relation to a mixture of different molecules refers to a process of removing at least some of the molecules from the mixture containing a desired molecule or set of molecules being purified. In many cases, solute not considered as part of the mixture being purified. The result is that the desired molecule of molecules will constitute a greater proportion of the molecules in the purified product mixture than in the original mixture. Thus, a “purified oligomer preparation” or “purified WP preparation” refer to a mixture or preparation which contains respectively oligomer or WP chain, and which contains a reduced fraction of other molecules which were previously present. For example, such purification may remove some or all of the other synthesis components and/or chains which do not have the intended length or contain other damage.

In the context of an oligomer application, the term “result” refers to the actual data or some aspect of the actual data which is directly generated by the practice of the application. As an example, the direct use of invention improved lower order application data in a higher order application will produce higher order application data which is improved in one or more aspects, such as accuracy or quantitation or reproducibility. In the same oligomer application context, the term “information” refers to some aspect of the conclusions (which may be overall conclusions) reached from or based on the application results or data. As an example, the use of invention improved lower order application results in a higher order application will produce higher order application information which is improved in one or more of interpretability or intercomparability or reliability or utility or predictive power.

In the context of oligomer sythesis and applications, the term “solid phase medium” refers to a material which is solid phase under the relevant conditions and to which oligomers can be directly or indirectly stably attached. Examples are well known, and include, for example, plastics (e.g., plates, slides, and chips), glasses (e.g., plates and slides), silicon chips, filters of various materials, and beads or other particles of various materials or combinations of materials.

Additional embodiments will be apparent from the Detailed Description and from the claims.

### DETAILED DESCRIPTION OF THE PREFERRED EMBODIMENTS

Contents

Solid-phase support used for oligomer synthesis.
The composition and length of the UP of the synthesized oligomers.
The creation and cleavage of the oligomer molecule oligomer cleavage site (OCS).
An example of a preferred practice of the invention.
Practice of the Invention which does not utilize a cleavage sensitive nucleotide or nucleotide substitute and which does not cleave the synthesized oligomer.
Practice of invention which uses multiple different invention-improved oligomers for the purpose of synthesizing a gene.
The invention-improved oligomer preparation “Invention Improvement Ripple Effect.”
Practice of the invention which uses one or more invention-improved oligomer preparations to produce invention-improved oligomer application methods and results: Zero Order Applications.
Practice of invention which produces improved methods and results for applications which utilize invention-improved oligomer application results of any kind: The Invention Improvement Ripple Effect.
Practice of the invention which produces improved methods and results for applications which utilize invention-improved first order application results or invention-improved higher order application results of any kind.
Conclusion

The invention provides a method and means for improving a chemically synthesized (CS) oligomer prep by significantly reducing the nucleotide sequence damage associated with the 3′ end portion of CS oligomer molecules, and also for improving the effectiveness and/or results of the application the CS oligomer is used in. Further, the invention provides a method and means for improving the enzymatic synthesis efficiency and quality of enzymatically synthesized (ES) nucleic acid molecules which are produced using CS oligomer primers. The method can be readily and inexpensively incorporated into current prior art practice production of CS oligomers and ES nucleic acid molecules of any kind.

A practice of the invention is the production of improved CS oligomer preparations which, relative to the usual prior art produced CS oligomer preparations, are associated with significantly lower amounts of nucleotide sequence damage in the 3′ end portion of the oligomer molecules. Another practice of the invention is the use of the invention-improved CS oligomer preps to produce CS oligomer application results which, relative to prior art produced CS oligomer application results, are improved CS oligomer application results. A further practice of the invention is the use of the invention-improved CS oligomer preps to produce CS oligomer applications which, relative to prior art application results, are more effective for the intended CS oligomer application uses. An additional practice of the invention is one or more CS oligomer synthesis production methods for producing the improved CS oligomer preps which, relative to prior art methods for producing such improved CS oligomer preps, is improved.

The prior art situation which gave rise to the invention, is that during the prior art process of synthesizing an intended oligomer the portion of the oligomer molecule which is synthesized earlier is associated with significantly more nucleotide sequence damage than the later synthesized nucleotide sequence. Another way of putting this is that for a prior art synthesized oligomer prep, the density of damaged nucleotides is greater for the earlier synthesized oligomer molecule portion, than the density of damaged nucleotides for the later synthesized oligomer potions. Here, the density of damaged nucleotides is expressed in terms of the relative number of damaged nucleotides present at each oligomer nucleotide sequence position for the oligomer prep.

The method of the invention improves this prior art situation by modifying the method of oligomer synthesis and processing. The general aspects of this modified method can be illustrated in terms of the synthesis of a CS oligomer with an intended nucleotide length N, of N=25, as follows, but clearly applies to synthesis of other oligomer lengths as well. In addition, while the following description includes a number of different steps or sub-processes, the method can also be carried out in syntheses which do not specifically include all of those steps or which combine certain steps.

(a) The synthesis process begins with the stepwise coupling of Z individual nucleotides, or nucleotide analogs, or nucleotide substitutes, to the solid support immobilized starting nucleotide, or nucleotide analog, or nucleotide substitute chain. Here Y designates the number of separate nucleotide or nucleotide analog or nucleotide substitute molecules present in the immobilized starting chain. For simplicity it will be assumed that Y=1 for the following discussion. Here Z designates the number of separate nucleotides or nucleotide analogs or substitutes coupled, and Z may be equal to one or more, but is preferably greater than 6. Note that the nucleotide sequence of this early synthesized sequence which is 1+Z nucleotides or nucleotide analogs or substitutes long, may or may not reflect a nucleotide sequence associated with the intended N=25 oligomer.

(b) To the growing oligomer chain, couple a specially designed nucleotide or nucleotide analog or nucleotide substitute molecule, which is herein termed a cleavage site compound or CSC. The site in a synthesized oligomer molecule sequence where the CSC exists is termed the oligomer cleavage site or OCS. The CSC molecule is designed so that under certain known conditions the CSC molecule becomes unstable, and one or more of the chemical bonds associated with the CSC is broken or cleaved, resulting in the breaking of the finished synthesized oligomer molecule backbone. The presence of a properly designed CSC molecule at a particular site in an oligomer molecule then, provides a specific oligomer cleavage site in the oligomer molecule which can be activated under certain known defined conditions. The use of such conditions results in the specific breaking of the oligomer phosphate backbone at the 5′ end of the the oligomer cleavage site or OCS. Note that one or more such CSC molecules can be coupled to each growing oligomer chain. Herein the number of CSC molecules added to the one growing chain is termed W, where W is equal to one or more. When more than one CSC molecule is coupled to an oligomer they can be grouped consecutively or put into separated sites. For the purposes of this general illustration, one CSC molecule will be added to the growing oligomer chain.

(c) Begin the synthesis of the intended N=25 oligomer molecule by coupling the intended oligomer's first 3′ end nucleotide to the CSC molecule coupled to the 1+Z growing oligomer chain. Complete the synthesis of the intended N=25 oligomer molecule.

(d) Upon completion of the oligomer synthesis, each completed oligomer molecule is composed of a (1+Z+W) portion and a portion representing the intended and wanted N=25 oligomer molecule. Herein the (1+Z+W) portion is termed the unwanted portion or UP, while the portion associated with the intended or desired oligomer is termed the wanted portion or WP. Note that while both the UP and WP of the finished oligomer molecules are associated with nucleotide sequence damage, the density of such damage is much higher for the UP than for the WP.

(e) The synthesized oligomer molecules undergo the steps of deprotection, removal from the solid surface, cleavage at the OCS, and separation of the UP molecule fraction from the WP molecule fraction. The order of performing these steps can vary depending on the overall design of the oligomer synthesis and processing system. Further, the separation process can be accomplished in a variety of ways to separate and purify the WP away from the UP and the deprotection products. Such a purified WP prep is here termed a “crude” WP oligomer prep. A “crude” WP oligomer prep is analogous to a prior art standardly synthesized “crude” oligomer prep. Such a crude WP oligomer prep consists of N−X, N, and at times N+X, WP oligomer molecules. Here X is equal to the difference in nucleotide length between the WP oligomer molecules with the intended nucleotide length N, and the actual nucleotide length of the WP oligomer molecule.

(f) The crude WP oligomer prep can be further purified and fractionated by standard prior art methods and processes. Note that oligomer preps prepared by such prior art methods and processes often contain significant amounts of N−X and/or N+X oligomers as well as the majority N oligomer molecules.

(g) The crude WP oligomer prep or further purified oligomer prep can then be used to produce improved ES oligomer and other nucleic acid molecule preps, or for other oligomer applications, to produce improved CS or ES oligomer application results.

(h) The above-described general illustration of the practice of the present invention has a large number of different specific permutations.

The method of the invention can be readily and simply incorporated into prior art oligomer synthesis and processing methods, and can be inexpensively accomplished.

A crude WP oligomer prep produced by the practice of the present invention is improved, relative to a prior art produced crude oligomer prep of the same intended oligomer, in the following ways. (i) The crude WP oligomer prep is associated with a significantly lower overall density of oligomer molecule nucleotide sequence damage, and a much lower density of oligomer molecule 3′ end nucleotide sequence damage. (ii) The crude WP oligomer prep is associated with a lower fraction of N−X oligomer molecules.

A further purified and fractionated WP oligomer prep produced by the practice of the invention is improved, relative to a prior art produced further purified and fractionated oligomer prep of the same intended oligomer, in the following ways. (a) The further purified WP oligomer prep is associated with a significantly lower overall density of oligomer molecule nucleotide sequence damage, and a much lower density of oligomer molecule 3′ end nucleotide sequence damage. (b) The further purified and fractionated WP oligomer prep is associated with a lower fraction of N−X oligomer molecules.

The practice of the invention then, provides a method and a means for eliminating from the produced WP oligomer prep the considerable oligomer nucleotide sequence damage which occurs during the early coupling steps of an intended oligomer chemical synthesis process. Note that virtually all prior art oligomer synthesis proceeds from the 3′ end to the 5′ end and that the starting nucleotide coupled to the CPG is the 3′ end nucleotide of the intended oligomer. Note further that the general practice of the invention is equally effective for oligomer synthesis methods which proceed from 5′ end to 3′ end. Here however the 5′ end portion is improved in nucleotide sequence damage and quality. For simplicity the 3′ to 5′ synthesized oligomers are emphasized herein, but the discussion will be directly applicable to the 5′ to 3′ synthesis situation.

The invention provides methods and means for producing crude oligomer preparations and fractionated purified oligomer preparations which, relative to prior art preparations of the same oligomer, are improved in quality. Further, the invention provides methods and means for producing crude oligomer prep and fractionated purified oligomer prep application results which, relative to prior art produced crude oligomer prep and fractionated purified oligomer application results, are improved.

A key aspect of the invention is the invention's methods and means for producing improved crude oligomer preparations for the intended oligomer. One or another of the methods and means of the invention for producing improved crude oligomer preparations for the intended oligomer, are readily incorporated into existing prior art synthesis systems and instruments of essentially all kinds. Such incorporation can be done inexpensively and effectively.

A very large number of different permutations of the methods and means of the invention for producing improved crude oligomer preps can be practiced. The choice of the preferred methods and means for the practice of the invention is influenced by the type and characteristics of the intended oligomer and its application. The preferred method and means of the invention will commonly reflect a compromise between effectiveness in producing improved crude oligomer preps, the ease of use associated with the method and means, and the expense involved in the practice of the invention. These issues will be discussed below, and a particularly advantageous practice of the invention will be described later.

Note that most prior art applications which utilize oligomer preps, utilize unpurified crude oligomer preps for the application. This occurs primarily because purification of the crude oligomer prep adds a significant mount of effort and expense to the oligomer prep. The invention provides a means for easily and inexpensively producing significantly improved crude oligomer preps.

Modifications of various aspects of the prior art oligomer synthesis reagents and processes which will facilitate the practice of the invention are discussed below. Such modifications are generally applicable for the low or high throughput production of improved crude oligomer preps of essentially all kinds.

Solid-Phase Support Used For Oligomer Synthesis

Prior art solid-phase supports are commonly composed of controlled pore glass (CPG) or plastic (3). For simplicity herein the solid-phase support will be termed CPG. However the discussion will apply directly to CPG, plastic and other solid-phase supports. For the prior art solid-phase synthesis of an intended oligomer, the intended oligomer's first 3′ end nucleotide is generally immobilized on the surface of the CPG. Therefore, for prior art oligomer synthesis the initial coupling step adds the oligomer's second 3′ end nucleotide to the CPG immobilized first 3′ end nucleotide. For the prior art synthesis of oligomers, which have different 3′ end nucleotides, different derivatized CPG preps must be prepared and used. For the prior art synthesis of oligomers in general, at least four different CPG preps, each containing a different immobilized nucleotide, are required. Further, for the synthesis of oligomers which have a modified 3′ end nucleotide, a differently derivatized CPG is required for each different modified 3′ end nucleoside. This requirement for multiple different derivatized CPGs adds significant complexity, effort, and expense, to the prior art intended oligomer synthesis process.

Prior art CPGs are generally designed so that the chemical bond which immobilizes the first 3′ end nucleoside to the CPG surface can be readily cleaved under specific conditions. This is done to facilitate the separation of the synthesized oligomer molecules from the CPG. Treating the CPG under specific conditions releases the synthesized oligomer molecules from the CPG surface and the CPG and the freed oligomers can then be readily separated from each other.

The just described prior art CPG practices can also be utilized in the practice of the invention for producing an improved crude preparation of the intended oligomer. However, the prior art CPG practice still requires multiple differently derivatized CPG preps. Further, while the prior art CPG practice facilitates the ease of separation of the synthesized oligomer from the CPG, it does not facilitate the separation of the invention produced UP and WP oligomer molecules. A modified prior art CPG practice is the preferred CPG practice for the invention. This preferred CPG practice is discussed below.

One aspect of the preferred CPG practice involves the type of nucleoside or nucleotide or nucleoside or nucleotide substitute which is immobilized on the CPG for the purpose of initiating the synthesis process. For the practice of the invention, the first 3′ end nucleoside does not need to be immobilized on the CPG because the UP of the synthesized oligomer is synthesized first. As a result, any nucleoside or nucleotide or substitute which enables the efficient initiation of the oligomer synthesis can be present on the CPG surface. The most effective nucleoside or nucleoside or substitute for this use can readily be determined by experimentation. The nature of such experimentation will be apparent to one of skill in the art. Therefore, only one type of CPG immobilized nucleoside or nucleoside or substitute is required for the synthesis of any oligomer or modified oligomer by the practice of the invention. This reduces the complexity, effort, and expense associated with oligomer synthesis. Note that different CPG preps may or may not be required for the synthesis of RNA and DNA oligomers. It should be possible to produce one CPG prep which can be used for either RNA or DNA synthesis.

A second aspect of the preferred CPG practice involves the stability of the attachment of the CPG immobilized nucleoside or nucleoside or substitute during the oligomer synthesis and post-synthesis de-protection and processing. For the preferred practice of the invention the attachment bond of the CPG immobilized nucleoside or nucleoside or substitute should be stable under all oligomer synthesis and oligomer processing conditions associated with the invention practice, including the oligomer deprotection and cleavage steps. Such a CPG immobilized nucleoside or nucleoside substitute stability, greatly facilitates the ease of separation of the WP of the synthesized oligomer from the CPG and the UP of the synthesized oligomer. This occurs because after specifically cleaving the synthesized oligomer at the OCS, the WP of the synthesized oligomer is no longer attached to the CPG, while the UP of the synthesized oligomer remains attached to the CPG. The WP can then be easily separated from the CPG-UP complex. A preferred practice of this preferred practice of the invention is one where the synthesized oligomer is completely de-protected while attached to the CPG and the freed protecting groups and reagents removed from the CPG-oligomer before the OCS cleavage occurs.

As will be apparent to one of skill in the art, many different preferred CPG practice effective permutations are possible.

The Composition and Length of the UP of the Synthesized Oligomer

For the practice of the invention the UP can be composed of one or more nucleotide types or nucleotide substitute molecule types. Further, the UP composition may include chemical functional groups or ligands or other molecules which can facilitate the separation of the UP and WP of the synthesized oligomer. For example, the UP may contain one or more biotin molecules and the WP none. Here the presence of the biotin in the UP can be used to facilitate the separation of the oligomer UP and WP.

An alternate UP composition may focus on reducing the cost of the UP as much as possible, and/or on reducing the synthesis time and or steps involved in synthesizing the UP. A further alternative is to produce bulk CPG preps which have the desired fully formed or partially formed UP immobilized on the CPG. These CPG-UP preps can then be used to produce the intended WP of the oligomer. In general the preferred UP composition will be the simplest most cost effective UP composition which will effectively accomplish the purpose of the designed UP. Note that the UP composition may or may not include nucleotides or modified nucleotides, and can consist of nucleotide substitute molecules of various kinds. As an example, properly modified sugar molecules could be used, and may in many ways be more effective than the nucleotides. One of skill in the art will be aware of many other possibilities. A large number of different possible effective UP compositions are possible.

For the practice of the invention, the UP length can vary greatly. The available prior art information indicates that even a 1+Z value of two will produce improved crude oligomer preps. This information indicates that the preferred 1+Z length ranges from about 6 to 20 or so nucleotides or nucleotide substitute molecules. For a particular oligomer synthesis system, the preferred length is the one which results in the maximum or near maximum reduction in the nucleotide sequence damage associated with the WP of the synthesized oligomer. Such a preferred length can be determined by experimentation.

The Creation and Cleavage of the Oligomer Molecule OCS

The coupling of the cleavage sensitive modified nucleotide or nucleotide substitute molecule onto the 1+Z growing oligomer chain provides a specific site in a completely synthesized oligomer molecule where the oligomer backbone or phosphate backbone can be broken to produce a UP oligomer molecule and a WP oligomer molecule. All or essentially all OCSs which are associated with the synthesized oligomer molecules should preferably be readily and completely cleaved under specific cleavage conditions which do not damage the WP oligomer, and which do not affect the immobilization of the UP to the CPG.

It is preferred that such cleavage occur as quickly and as completely as possible, under conditions which are as mild as possible. Further it is preferred that the coupling of the cleavage sensitive nucleotide or nucleotide substitute be easily incorporated into existing oligomer synthesis methods and processes. Such cleavage sensitive nucleotides or nucleotide substitutes are routinely incorporated into growing oligomer chains by the prior art. As an example prior art syntheses often incorporate into a growing oligomer chain an apurinic nucleotide or nucleotide substitute. The process of coupling the cleavage sensitive nucleotide or nucleotide substitute can be easily integrated into existing oligomer synthesis methods, protocols, and processes. It is also preferred that the cleavage process does not result in a significant chemical modification of the separated WP of the oligomer, and that the chemical configuration of the 3′ end of the WP oligomer is appropriate for the intended WP oligomer use.

Prior art oligomer synthesis practice has developed cleavage sensitive nucleotides or nucleotide substitutes which are often incorporated into growing oligomer chains by standard prior art oligomer synthesis methods. As an example, prior art often couples an apurinic nucleotide substitute into a growing oligomer chain. The OCS created by the presence of such an apurinic nucleotide substitute in an oligomer molecule can be readily and specifically cleaved under well known basic conditions.

One of skill in the art will recognize that a wide variety of nucleotide or nucleotide substitute cleavage sensitive molecules and cleavage conditions can be produced and used for the practice of the invention, and that the UP may possess a non-phosphate backbone and that the link between the CSC and the first 3′ nucleotide of the WP can be other than a phosphate bond.

As discussed, it is preferred that the CPG attached synthesized oligomer molecules be completely deprotected and the freed protection groups separated from the CPG+UP+WP synthesized oligomer complex before the cleavage step is done. Then, after the cleavage step the CPG•UP oligomer complex can be readily separated from the WP oligomer molecules. The crude WP oligomer prep can then be processed for further purification or for use in its intended application.

An Example of A Preferred Practice of the Invention

A large number of possible permutations of the practice of the invention can be utilized. Following is a description of the practice of the invention which is, in the context of the use of known prior art reagents and methods and process, a preferred practice of the invention. This description is presented in terms of the chemical synthesis of an intended DNA oligomer. However, it will be apparent to one of skill in the art how to apply the basic aspects of the invention to the synthesis of RNA or other nucleic acid oligomers. The description follows.

(a) Produce CPG which has immobilized on its surface thymidine nucleoside molecules which can be used to initiate the chemical synthesis of the intended oligomer. The immobilized thymidine nucleoside-CPG surface bond is stable under all conditions used for the synthesis and processing of the oligomer. This can be accomplished using known prior art CPG production methods. Thymidine nucleotide was chosen because it is a simple nucleoside and the thymine base does not require protection during synthesis. The thymidine CPG is used for the chemical synthesis of the intended oligomer.

(b) Begin the synthesis of the UP of the oligomer. By standard prior art methods synthesize the 1+Z oligomer portion where Z represents 10 thymidine nucleotide molecules which are added to each growing oligomer chain.

(c) To each 1+Z growing oligomer chain molecule couple one cleavage sensitive nucleotide or non-nucleotide molecule by standard prior art methods. Such cleavage sensitive nucleotide or non-nucleotide compounds which are suitable for use in standard prior art oligomer synthesis can be produced.

(d) Starting with the coupling to the cleavage sensitive nucleotide of the 3′ end nucleotide of the intended oligomer, synthesize the intended oligomer molecule using standard prior art oligomer synthesis methods. During the synthesis of the WP of the oligomer, a capping step is preferred for each coupling step. At the completion of the oligomer synthesis the synthesized oligomer molecule attached to the CPG is composed of a UP and WP.

(e) The completed oligomer molecules attached to the CPG are then deprotected by a standard prior art treatment. After this deprotection step, the protective groups are no longer attached to the UP+WP oligomer, which is still attached to the CPG.

(f) The CPG and attached UP+WP oligomer is then separated from the released protective and other unwanted groups in the oligomer preparation.

(g) The CPG attached UP+WP oligomer molecules are then cleaved at the OCS. After cleavage the UP is still attached to the CPG while the WP is no longer attached to the CPG.

(h) The WP oligomer is then separated from the CPG+UP oligomer complex to produce a purified crude WP oligomer preparation.

(i) If desired, the crude WP preparation can be further fractionated by standard methods to produce a purified N WP oligo preparation.

Practice of the Invention Which Does Not Utilize A Cleavage Sensitive Nucleotide or Nucleotide Substitute and Which Does Not Cleave the Synthesized Oligomer

Many prior art oligomer applications synthesize oligomers on a solid surface and do not remove the deprotected oligomers from the solid surface for use, since the oligomer application utilizes surface immobilized oligomers. An example of such an application is the oligonucleotide microarrays where the oligomers are synthesized on the microarray device surface (5). For such applications the crude oligomer prep consists of the deprotected oligomer molecules which remain attached to the surface after removing the freed protective groups. This immobilized crude oligomer prep is also known to be associated with a much higher nucleotide sequence damage density at the 3′ end of the oligomer. The intended nucleotide length N of such surface synthesized and used oligomer molecules is generally around 20 to 25 nucleotides, but the intended N can be as high as 60 to 70 nucleotides or higher.

Here a modified practice of the invention can be used to obtain a crude WP oligomer preparation which, relative to prior art produced crude oligomer preparations of the same oligomer represented by the WP oligomer, has an overall significantly lower nucleotide sequence damage density and a much lower 3′ end nucleotide sequence damage density, and is therefore significantly improved. The general aspects of such a modified practice of the invention are described below. The general description will enable one of skill in the art to practice one or many forms of the modified practice of the invention. Such a modified practice of the invention can be practiced using existing oligomer synthesis methods. The general description follows.

(a) Using standard prior art synthesis methods initiate the oligomer synthesis at the 3′ end of the oligomer chain and stepwise couple Z nucleotides or nucleotide substitutes to form a growing chain Z nucleotides or nucleotide substitutes in length, where Z is preferably equal to at least 6 or more nucleotides, nucleotide analogs, or nucleotide substitutes. This Z long growing chain is equivalent to the UP. Preferably, the UP should be composed of Z nucleotide substitutes which cannot base pair with natural or modified nucleotides and which are not chemically reactive or sticky.

(b) To the 5′ end of the growing Z chain couple the first 3′ end nucleotide of the desired intended oligomer, and then complete the synthesis of the intended oligomer, e.g., by standard prior art methods. The portion of the finished synthesized oligomer which represents the intended desired oligomer is equivalent to the oligomer WP.

(c) Deprotect the synthesized oligomers and separate the released protective groups away from the immobilized synthesized crude oligomer preparation.

(d) For this immobilized crude oligomer preparation, the WP of the oligomer is associated with significantly less nucleotide sequence damage than prior art produced immobilized crude oligomer preparation of the same intended oligomer, and no CSC is needed for the practice of the invention.

Practice of the Invention which uses Multiple Different Invention-improved Oligomers for the Purpose of Synthesizing a Gene

Prior art gene synthesis practitioners often combine multiple different unpurified and purified oligomer preps in order to synthesize genes of known nucleotide sequence in a test tube. Prior art has utilized this approach to synthesize a viral chromosome (12), and plans to synthesize a small bacterial chromosome. Such a synthesis often utilizes both CS and ES nucleic acids, and almost always uses crude CS oligomer preps. The synthesis of a gene with a known nucleotide sequence from different CS oligomer molecules is complicated by the various forms of nucleotide sequence damage associated with each CS crude or purified oligomer prep. Significantly reducing the amount of nucleotide sequence damage which is associated with a CS oligomer prep will result in a significantly improved gene synthesis method. Thus, the use of invention-improved CS or ES oligomer molecules in the gene or chromosome synthesis method will result in reducing the amount of the nucleotide sequence damage associated with the combined oligomer and produce an invention-improved gene synthesis method, and is a practice of the present invention.

The Invention-Improved Oligomer Preparation “Invention Improvement Ripple Effect”

The production and use of an invention-improved oligomer prep causes an “Invention Improvement Ripple Effect” which extends far downstream from the direct use of an improved oligomer prep in its particular oligomer application. Once an invention-improved oligomer prep is produced, the immediate and direct downstream use of the improved oligomer is to use the invention-improved oligomer prep in an oligomer application to produced improved oligomer application results. Such an oligomer application is termed a “zero order application”. A downstream invention improvement ripple effect is the use of the improved zero order application results in further applications to produce improved further application results. Herein, the direct use of improved zero order oligomer application results in a further application is termed a “first order application”. A still further downstream improvement ripple effect is the use of the improved first order application results in a still further application to produce improved still further application results. Herein, the direct use of improved first order application results for a still further application is termed a “second order application”. In the same vein then, the use of improved second order application results in a third order application produces improved “third order application” results. In this same vein, improved fourth order application results, improved fifth order application results, etc., can be generated.

Each invention improvement ripple effect begins with the production of one or more invention-improved oligomer preparations. The production of each separate improved first, second, third, fourth etc order application result is a practice of the invention. This will be discussed below.

Practice of the Invention Which Uses One or More Invention-Improved Oligomer Preparations to Produce Invention-Improved Oligomer Application Methods and Results: Zero Order Applications

The vast majority of prior art oligomer application methods are developed and optimized using chemically synthesized oligomer preparations which are produced by standard 3′ to 5′ prior art chemical synthesis methods. As discussed, such oligomer preparations are very often associated with significant nucleotide sequence damage in the 3′ end portion of the oligomer. While standard prior art chemical synthesis methods start the synthesis at the 3′ end of the oligomer. A seldom used prior art chemical synthesis method is to begin synthesis at the 5′ end of the oligomer. Here, the significant nucleotide sequence damage is associated with the 5′ end of the oligomer. For simplicity, the 3′ to 5′ standard synthesis is emphasized herein, but the discussion is generally directly applicable to the rare 5′ to 3′ synthesis of oligomer preparations.

Because the prior art oligomer preps used to develop and optimize oligomer application methods are generally significantly damaged, the resulting prior art developed and optimized oligomer application methods are sub-optimal in development and performance. The use of such sub-optimal oligomer application methods then produces sub-optimal oligomer application results. Clearly, the higher the quality of the oligomer prep used for an oligomer application method's development, optimization and use, the more optimal the development and performance and use of the oligomer application method, and the more improved the oligomer application results. Thus, the use of the invention-improved oligomer preparations for the development, optimization, and use of an oligomer application method will produce an oligomer application method which is significantly improved in optimization and performance relative to prior art produced oligomer application methods, and which produces oligomer application results which are significantly improved relative to prior art oligomer application results, and is therefore a practice of the present invention.

An example of a zero order application follows. Almost always the prior art uses 3′ to 5′ synthesized oligomer preparations for the development and optimization of oligomer primers used for the in vitro enzymatic synthesis of nucleic acids. Such in vitro enzymatic synthesis methods comprise DNA and RNA synthesis methods of all kinds, including PCR and cDNA synthesis methods, and various RNA synthesis methods such as those based on T₇ and other promoters (7).

The 3′ end portion of an oligomer primer molecule is the “Action” end of the primer molecule because enzymatic DNA and RNA synthesis begins at the 3′ end of the oligomer primer molecule. The enzymatic synthesis efficiency of an oligomer primer molecule is significantly reduced by the presence of significant 3′ end nucleotide sequence damage (11). Thus, the use of prior art produced oligomer primer preps for the development and optimization of an enzymatic synthesis method, will produce a prior art oligomer primer application in vitro synthesis which is sub-optimal in development, performance and application result quality, relative to an invention-improved oligomer primer application in vitro synthesis method which is significantly improved in development, performance, and application result quality.

The above example of the use of invention-improved oligomers to produce improved oligomer application method and improved oligomer application results, is only one of a large number of different possible zero order applications for invention-improved oligomer preps. One of skill in the art will recognize that the invention can be applied in a similar way for a large variety of different oligomer applications. These include but are not limited to oligomer applications for: site directed mutagenesis; capture probes for microarrays and other gene expression analysis applications; DNA and/or RNA hybridization probes for PCR and RT-PCR; genetic gengineering of proteins' DNA and/or RNA probes for diagnostics; siRNA, miRNA and other regulatory RNAs; molecular beacons and other fluorescently labeled DNA or RNA probes; single nucleotide polymorphism or mutation detection; oligomer related thermodynamic data determination; gene cloning; nucleic acid sequencing; nucleic acid standards; gene synthesis; and others.

Practice of Invention Which Produces Improved Methods and Results for Applications Which Utilize Invention-Improved Oligomer Application Results of Any Kind: First Order Applications

The direct use of invention-improved zero order application results in an oligomer application of any kind is termed a first order application. Such a use produces an invention-improved first order application method and improved first order application results which are, relative to prior art produced first order application methods and results, significantly improved, and is a practice of the present invention.

An example of the production of improved first order application methods and results utilizes the above described example of obtaining an invention-improved oligomer primer for enzymatic nucleic acid synthesis application results. In vitro enzymatic DNA and RNA synthesis oligomer primer applications are central to most present-day gene expression analyses and gene expression comparison analyses. Deficiencies in the synthesis efficiency of a gene expression assay often cause the assay measured mRNA abundance and gene expression ratio values to deviate significantly from biological accuracy. For RT-PCR assays significant 3′ end oligomer primer nucleotide sequence damage can greatly affect the efficiencies of cDNA synthesis and PCR amplicon DNA synthesis, and cause them to be very significantly sub-optimal. Thus, the use of prior art oligomer primers in an RT-PCR assay will produce sub-optimal gene expression analysis results. Such prior art RT-PCR sub-optimal results are often used in a prior art first order application, as for example a data mining analysis application. The result is a significantly sub-optimal data mining analysis result. The use of invention-improved in vitro enzymatic nucleic acid synthesis oligomer primer application results in a data mining or other first order application, then produces data mining and other first order application methods which are significantly improved relative to prior art first order application methods, and data mining and other first order application results which are significantly improved relative to prior art produced first order application results.

The above described example of the use of invention-improved oligomer application results to produce significantly improved first order application methods and significantly improved first order application results, is only one of many different possible uses of improved oligomer application results for improving first order application methods and results. One of skill in the art will recognize that a large variety of such first order applications of improved oligomer application results exist. Further, one of skill in the art will recognize that the use of relevant improved results in an application of any kind will improve the results of the application of any kind, relative to the results produced by the application of any kind which utilizes prior art relevant results which are not improved. One of skill in the art will also recognize that the improved oligomer application results which can be utilized for one or more first order applications include, but are not limited to improved results for the following oligomer applications: (a) site directed mutagenesis; (b) capture probes for Microarray and other gene expression analysis; (c ) DNA or RNA hybridization probes for PCR and RT-PCR and diagnostic assays; (d) genetic engineering of proteins; (e) gene cloning; (f) siRNA, miRNA and other Regulatory nucleic acids; (g) single nucleotide polymorphism and mutation detection; (h) nucleic acid sequencing; (i) thermodynamic data determination; (j) nucleic acid standards; (k) RNA amplification methods.

Practice of the Invention Which Produces Improved Methods and Results for Applications Which Utilize Invention-Improved First Order Application Results or Invention-Improved Higher Order Application Results of Any Kind

An application method of any kind which directly utilizes the results of a first order application method is herein termed a second order application method, while an application method which utilizes the results of a second order application method is termed a third order application. The further use of second order or third order or fourth order etc application method results is herein termed a higher order application method. The use of invention-improved first order application results in a second order application method produces improved second order application methods and improved second order application results, and is a practice of the invention. Similarly, the use of invention-improved second order application results in a third order application method produces improved third order application methods and improved third order application results, and is a practice of the invention. This same basic rationale concerning the use of improved application results which derives from one or more invention-improved oligomer preps allows the production of invention-improved higher order application results beyond third order results. This discussion highlights the reality that producing an invention-improved oligomer prep also improves the information obtained when that improved oligomer prep is used in an oligomer application and when the improved oligomer application result is used as part of further applications.

An example of producing invention-improved results for a second order application is the use of an above discussed invention-improved first order application result or data mining analysis result in a second order application or a systems biology analysis application. An example of producing invention-improved results for a third order application is the use of the invention-improved second order application results and/or systems biology analysis results, in a third order application whose purpose is to select drug candidate genes for pharmaceutical development. The invention-improved third order application or drug candidate selection application results could then be used in a fourth order application, whose purpose is to identify the drug candidates market population, to produce invention-improved fourth order application results, and so on.

One of skill in the art will recognize the large number of possibilities which exist for first, second, third, and higher order application combinations for each improved oligomer prep and improved oligomer application result and that many of these possibilities and combinations have direct utility and application to many basic and applied and industrial research and development and manufacturing applications of many kinds and more specifically to the overall process of pharmaceutical drug discover, drug development, drug validation, drug characterization, drug manufacturing, drug use and marketing.

CONCLUSION

For the purpose of explanation the foregoing discussions used specific nomenclature to provide a thorough understanding of the invention and its many embodiments. However, it will be apparent to one of skill in the art that this nomenclature and description are but one way to describe the invention and its mode of practice. Thus, the foregoing nomenclature and description are presented for the purpose of illustration and description, and they are not intended to be exhaustive or to limit the invention to the precise forms disclosed, and obviously many modifications and variations are possible as a result of the above techniques. The discussions presented were selected and described in order to best explain the present invention and its practical applications, and to thereby enable others skilled in the art to best practice the invention and various embodiments with various modifications, as are suited to the particular use contemplated.

References

1) Herdewin. Oligonucleotide Synthesis. Methods and Applications. Methods in Mol. Bio. Vol 288. Humana Press (2005).

2) Khudyakov et al. Artificial DNA. Methods and Applications. CRC (2003)

3) Agrawal. Protocols for Oligonucleotides and Analogs. Synthesis and Properties. Methods in Mol. Bio. Vol 20. Humana Press (1993).

4) Agrawal. Protocols for Oligonucleotide Conjugates. Synthesis and Analytical Techniques. Methods in Mol. Bio. Vol 26. Humana Press (1994).

5) McGall et al. The Efficiency of Light-Directed Synthesis of DNA Arrays on Glass Substrates. J. Am. Chem. Soc. 119(22): 5081-5090 (1997).

6) Demidov et al. DNA Amplification. Current Technologies and Applications.

Horizon Bioscience (2004).

7) Botwell et al. DNA Microarrays. Cold Spring Harbor Lab Press (2003).

8) Becker et al. Error Analysis of Chemically Synthesized Polynucleotides. Biotechniques. 24(2): 256-260 (1998).

9) Temsamani et al. Sequence Identity of the N-1 Product of a Synthetic Oligonucleotide. Nuc. Acid Res. 23(11): 1841-1844 (1995).

10) Chen et al. Analysis of Internal N-lmer. Deletion Sequences in Synthetic Oligodeoxyribonucleotides by Hybridization to an Immobilized Probe Array. Nuc. Acid Res. 27(2): 389-395 (1999).

11) Kaltenbock et al. Differential Amplification Kinetics for Point Mutation Analysis by PCR. Biotechniques. 24(2): 202-206 (1998).

12) Smith et al. Generating a Synthetic Genome by Whole Genome Assembly:phix 174 Bacteriophage from Synthetic Pligonucleotides. Proc. Natl. Acad. Sci. (USA) 100(26):15440-15445(2003)

All patents and other references cited in the specification are indicative of the level of skill of those skilled in the art to which the invention pertains, and are incorporated by reference in their entireties, including any tables and figures, to the same extent as if each reference had been incorporated by reference in its entirety individually. The citation of any publication for its disclosure prior to the filing date should not be construed as an admission that the present invention is not entitled to antedate such publication by virtue of prior invention.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the sequences of oligomers and to the moieties used in synthesizing those oligomers. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention.

Thus, additional embodiments are within the scope of the invention and within the following claims.

1. A method for chemically synthesizing a nucleotide oligomer preparation significantly improved in nucleotide sequence quality, comprising
sequentially coupling in order chain moieties of a wanted polymer (WP) nucleotide sequence to an immobilized unwanted polymer (UP) comprising in order an attachment chain Y chain moieties in length, a spacer chain Z moieties in length, and optionally W cleavage site compound (CSC) chain moieties, wherein Y,Z, and W are each independently equal to or greater than 1, forming a UP+WP chain.

2. The method of claim 1, further comprising specifically cleaving WP from UP at one or more cleavage site chain moieties.

3. The method of claim 2, further comprising separating WP from UP.

4. The method of claim 1, further comprising sequentially coupling Z nucleotides, nucleotide analogs, nucleotide substitues, or a combination thereof to a surface immobilized attachment chain Y chain moieties in length of nucleotides, nucleotide analogs, nucleotide substitutes, or a combination of such molecules to form an attachment spacer chain; and
coupling to the attachment spacer chain W cleavage site compounds (CSC) producing said unwanted polymer (UP).

5. The method of claim 2, wherein said cleavage is performed by subjecting the UP+WP chain to a condition or combination of to or more conditions which promote the specific cleavage of the UP+WP chain at the cleavage site compound location to convert the intact UP+WP finished oligomer chain to UP and WP oligomer molecules which are not attached together.

6. The method of claim 1, further comprising subjecting the WP to one or more conditions which promotes the removal of all or essentially all chemical synthesis process-related protective and modifier chemical groups from the QP, or optionally from both the UP and WP.

7. The method of any of claim 2, further comprising purifying the WP away from the UP and other unwanted non-WP molecules and solid components.

8. The method of claim 2, further comprising processing the WP to produce a purified preparation of WP enriched for WP oligomer molecules of the intended nucleotide length N.

9. The method of claim 1, wherein,
Y is 1 or 2.

10. The method of claim 1, wherein Z is equal to 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 nucleotides, nucleotide analogs, or nucleotide substitues, or combinations thereof.

11. The method of claim 1, wherein Z is 2-50 inclusive, or is any integer value in that range.

12. The method of claim 1, wherein Z is 2-50 inclusive, or is any integer value in that range.

13. The method of claim 1, wherein W is equal to 1 or 2 or 3 or 4 or 5 or more.

14. The method of claim 2, wherein the 3′ end nucleotide of a 3′ to 5′ synthesized and cleaved and deprotected WP oligomer, or the 5′ end nucleotide of a 5′ to a 3′ synthesized and cleaved and deprotected WP, possesses the proper chemical composition and configuration so that the WP oligomer can be directly used in the intended oligomer application without further modification.

15. The method of claim 1, wherein the starting nucleotide, nucleotide analog, or nucleotide substitute molecules are attached to the surface by a chemical bond which is not cleaved under any of the conditions used for the production or use of the purified crude WP oligomer prep.

16. The method of claim 1, wherein the starting nucleotide, nucleotide analog, or nucleotide substitute molecules are attached to the surface by a chemical bond which is not cleaved under any of the conditions used for the production or use of the purified crude WP oligomer prep.

17. The method of claim 1, wherein, the starting claim of nucleotide or nucleotide substitution molecules is attached to the surface by a chemical bond which does not cleave under any of the conditions used for the UP or WP synthesis or processing or use; and
a cleavage site compound (CSC) molecule is not incorporated into the UP oligomer chain, such that after deprotecting the UP+WP oligomer molecules and removing all or essentially all of the chemical synthesis process related protective and modifier chemical groups from the UP+WP oligomer molecule, and the UP+WP oligomer molecules remain stably attached to the surface of the synthesis support.

18. The method of claim 17, where said oligomer preparation is used in an oligomer application with said UP+WP chains in the immobilized state.

19. The method of claim 1, wherein a modified or unmodified RNA or DNA QP oligomer is produced.

20. The method of claim 1, wherein a chimeric WP oligomer consisting of a combination of modified RNA and modified or unmodified DNA is produced.

21. The method of claim 1, wherein the starting chain contains at least one nucleotide or nucleotide analog or nucleotide substitute molecules, or a combination of 1 or 2 or more of said nucleotide, nucleotide analog, nucleotide substitue molecules.

22. A method for producing improved oligomer primer dependent in vitro enzymatically synthesized RNA or DNA molecules, comprising
utilizing one or more oligonucleotides from an improved oligomer preparation prepared according to claim 1 as primers in an in vitro enzymatic synthesis system to produce improved enzymatically synthesized RNA (ES RNA) or DNA (ES DNA) molecules.

23. (canceled)

24. (canceled)

25. An improved oligonucleotide preparation, comprising a set of chemically synthesized immobilized oligonucleotide chains, said chains comprising an unwanted portion chain (UP) at least 3 chain moieties in length; and a wanted chain consisting primarily of a desired sequence of nucleotides or nucleotide analogs or both, wherein said unwanted portion chain (UP) comprises at least one cleavage site moiety proximal to said wanted chain.

26. The oligonucleotide preparation of claim 25, where the first 10 nucleotides or nucleotide analog residues of said wanted chains contain an average density of damaged nucleotide sequence site which is no more than 0.9 times the average density of damaged nucleotide sequence sites in the first ten nucleotide or nucleotide analogs in the same sequence produced in the absence of unwanted chains.

27. The oligonucleotide preparation of claim 25, wherein the first 10 nucleotides or nucleotide analog residues of the N-X fraction molecules in said wanted chains contain an average density of damaged nucleotide sequence sites which is not more than 0.8 times the average density of damaged nucleotide sequence sites in the first ten nucleotide or nucleotide analog residues in the same sequence oligomer preparation produced in the absence of unwanted chains.

28. The oligonucleotide preparation of claim 25, wherein said unwanted chain is 4-40 chain moieties in length.

29. The oligonucleotide preparation of claim 25, wherein said unwanted chain is 6-30 chain moieties in length.

30. (canceled)

31. (canceled)

32. (canceled)

33. A kit for preparing an oligonucleotide preparation, comprising at least one solid phase medium having attached thereto a plurality of immobilized oligomers each having an attached terminus and a free terminus, said oligomers comprising in order an attachment moiety, a spacer chain, and a cleavage site moiety, wherein the free terminus of each said chain is functionalizing or is chemically suitable for functionalizing with a functional group for extending the oligomer with a plurality of chain moieties.

34. The kit of claim 33, wherein said solid phase medium comprises beads.

35. The kit of claim 33, wherein said solid phase comprises a chip.

36. The kit of claim 33, wherein said solid phase comprises a plate.

37. The kit of claim 33, wherein said solid phase comprises a filter.

38. The kit of claim 33, further comprising instructions for use of said solid phase medium.

39. (canceled)

40. The kit of claim 33, further comprising separate quantities of phosphoramidites corresponding to each of 4 different naturally occurring ribonucleotides or deoxyribonucleotides.

41. (canceled)

42. The kit of claim 33, wherein said oligomers consist essentially of 6-40 chain moieties.

43. The kit of claim 33, wherein said oligomers consist essentially of 10-30 chain moieties.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. A method for producing improved oligomer application information and results for a zero order oligomer application, which directly utilizes present invention-improved oligomer preparations, comprising
using the method of one ore more of claims 1-21 to produce an improved oligomer preparation, and
then utilizing the improved oligomer preparation in a zero order application, thereby, producing one or more improved zero order application information and results.

60. The method of claim 59, wherein the zero order application comprises an application selected from the group consisting of,
a) a primer application;
b) a hybridization probe application;
c) a gene expression analysis application;
d) a site directed mutagenesis application;
e) a microarray hybridization capture probe application;
f) a cloning application;
g) a gene synthesis application;
h) a fluorescent-labeled oligomer application;
i) a mutation or single nucleotide polymorphism detection application;
j) a nucleic acid sequencing application;
k) a nucleic acid standard application; and
l) a production of an oligomer primer dependent in vitro enzymatically synthesized RNA (ES RNA) or DNA (ES DNA) molecule application.

61. (canceled)

62. (canceled)

63. (canceled)

64. (canceled)

65. (canceled)

66. (canceled)

67. (canceled)
